SEQUENCE LISTING (5 sequences)

SEQ ID NO: 1 - protein, 159 aa, Betula pendula
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 2 - protein, 5 aa, Artificial Sequence
  Synthetic
ARRXR

SEQ ID NO: 3 - protein, 4 aa, Artificial Sequence
  Synthetic
RRXS

SEQ ID NO: 4 - protein, 5 aa, Artificial Sequence
  Synthetic
RRXXS

SEQ ID NO: 5 - protein, 9 aa, Artificial Sequence
  Synthetic
XXXXXXXXY

(19) United States
(12) Patent Application Publication
(10) Pub. No.: US 2003/0041354 A1
(43) Pub. Date: Feb. 27, 2003

(54) Transgenic plants

(75) Inventors: Soren Kjaerulff, Vanlose (DK); Erwin Ludo Roggen, Lyngby (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 09/981,009

(22) Filed: Oct. 16, 2001

Related U.S. Application Data
(60) Provisional application No. 60/241,870, filed on Oct. 20, 2000.

(30) Foreign Application Priority Data
Oct. 17, 2000 (DK) 2000 01546

(51) Int. Cl. A01H 5/00; G01N 33/53; C12N 5/04

(52) U.S. Cl. 800/288; 435/7.1; 530/388.1; 435/419

(57) ABSTRACT

The present invention relates to a method of producing a transgenic plant expressing a protein having modified immunogenicity as compared to the parent protein, a transgenic plant expressing said protein, which is less immunogenic as compared to the non-transgenic plant.

### CROSS-REFERENCE TO RELATED APPLICATIONS

This application claims, under 35 U.S.C. 119, priority or the benefit of Danish application no. PA 2000 01546 filed Oct. 17, 2000 and U.S. provisional application No. 60/241,870 filed Oct. 20, 2000, the contents of which are fully incorporated herein by reference.

### FIELD OF INVENTION

The present invention relates to a method of producing a transgenic plant expressing a protein having modified immunogenicity as compared to the parent protein, a transgenic plant expressing said protein, which is less immunogenic as compared to the non-transgenic plant.

### BACKGROUND OF THE INVENTION

Today many individuals including humans and animals are suffering from allergic diseases. Allergies exist to many different substances such as to foods, grasses, trees and insects.

Depending on the application, individuals get sensitized to the respective allergens by inhalation, direct contact with skin and eyes, or injection. The general mechanism behind an allergic response is divided in a sensitization phase and a symptomatic phase. The sensitization phase involves a first exposure of an individual to an allergen. This event activates specific T- and B-lymphocytes, and leads to the production of allergen specific IgE antibodies (in the present context the antibodies are denoted as usual, i.e. immunoglobulin E is IgE etc.). These IgE antibodies eventually facilitate allergen capturing and presentation to T-lymphocytes at the onset of the symptomatic phase. This phase is initiated by a second exposure to the same or a resembling antigen. The specific IgE antibodies bind to the specific IgE receptors on mast cells and basophils, among others, and capture at the same time the allergen. The polyclonal nature of this process results in bridging and clustering of the IgE receptors, and subsequently in the activation of mast cells and basophils. This activation triggers the release of various chemical mediators involved in the early as well as late phase reactions of the symptomatic phase of allergy. Prevention of allergy in susceptible individuals is therefore a research area of great importance.

Various attempts to reduce the immunogenicity of polypeptides and proteins have been conducted. It has been found that small changes in an epitope may affect the binding to an antibody. This may result in a reduced importance of such an epitope, maybe converting it from a high affinity to a low affinity epitope, or maybe even result in epitope loss, i.e. that the epitope cannot sufficiently bind a B-cell to elicit an immunogenic response.

In WO 99/53038 (Genencor Int.) as well as in prior references (Kammerer et al, Clin. Exp. Allergy, 1997, vol. 27, pp 1016-1026; Sakakibara et al., J. Vet. Med. Sci., 1998; vol. 60, pp. 599-605), methods are described, which identify linear T-cell epitopes among a library of known peptide sequences, each representing part of the primary sequence of the protein of interest. Further, several similar techniques for localization of B-cell epitopes are disclosed by Walshet et al, J. Immunol. Methods, vol. 121, 1275-280, (1989), and by Schoofs et al. J. Immunol. vol. 140, 611-616, (1987). These methods, however, only leads to identification of linear epitopes, not to identification of ‘structural’ or ‘discontinuous’ epitopes, which are found on the 3-dimensional surface of protein molecules and which comprise amino acids from several discrete sites of the primary sequence of the protein. For several allergens, it has been realized that the dominant B-cell epitopes are of such discontinuous nature (Collins et al., Clin. Exp. All. 1996, vol. 26, pp. 36-42).

In WO 92/10755 a method for modifying proteins to obtain less immunogenic variants is described. Randomly constructed protein variants, revealing a reduced binding of antibodies to the parent enzyme as compared to the parent enzyme itself, are selected for the measurement in animal models in terms of allergenicity. Finally, it is assessed whether reduction in immunogenicity is due to true elimination of an epitope or a reduction in affinity for antibodies. This method targets the identification of amino acids that may be part of structural epitopes by using a complete protein for assessing antigen binding. The major drawbacks of this approach are the ‘trial and error’ character, which makes it a lengthy and expensive process, and the lack of general information on the epitope patterns. Without this information, the results obtained for one protein cannot be applied on another protein.

WO 99/47680 (ALK-ABELLÓ) discloses the identification and modification of B-cell epitopes by protein engineering. However, the method is based on crystal structures of Fab-antigen complexes, and B-cell epitopes are defined as “a section of the surface of the antigen comprising 15-25 amino acid residues, which are within a distance from the atoms of the antibody enabling direct interaction” (p.3). This publication does not show how one selects which Fab fragment to use (e.g. to target the most dominant allergy epitopes) or how one selects the substitutions to be made. Further, their method cannot be used in the absence of such crystallographic data for antigen-antibody complexes, which are very cumbersome, sometimes impossible, to obtain—especially since one would need a separate crystal structure for each epitope to be changed.

There is a need for methods to create foods which are less allergenic by identifying epitopes on proteins and alter these epitopes in order to modify the immunogenicity of proteins in a targeted manner and transforming the food material with cloned expression vectors of the modified protein. While the technology to make genetically engineered plant and animals is at this point well established, useful modifications would require understanding how allergens can be modified so that they retain the essential functions for the plants nutritional value, taste characteristics, etc., but no longer elicit as severe an allergic response.

WO 99/38978 describes a method of making a modified allergen which is less reactive with IgE. The IgE binding sites can be converted to non-IgE binding sites by masking the site with a compound that prevent IgE binding or by altering a single amino acid within the protein. It is desirable to modify allergens to diminish binding to IgE while retaining their ability to activate T cells. The reference also describes a transgenic plant or animal expressing the modified allergen said plant or animal eliciting less of an allergic response than the natural organisms.

WO 01/49830 (unpublished at the priority date of the present invention) describes modified potato protein patatin having reduced allergenicity and presents a method for identifying linear epitopes on the protein as a target for modification using synthesized peptides. WO 01/49830 also describes transformed plants.

Hence, it is of interest to establish a general and efficient method to identify structural epitopes on the 3-dimensional surface of environmental allergens, modifying the allergens and transforming a plant with the modified protein thereby making the plant less allergenic as compared to the plant not transformed with the modified allergens.

### SUMMARY OF THE INVENTION

The present invention relates to a method of producing a plant expressing a protein variant having modified immunogenicity as compared to a parent protein, comprising the steps of:

(a) obtaining antibody binding peptide sequences involved in antibody binding,

(b) using the sequences to localize epitope sequences on the primary and/or the 3-dimensional structure of a parent protein,

(c) defining an epitope area including amino acids situated within 5 Å from the epitope amino acids constituting the epitope sequence,

(d) changing one or more of the amino acids defining the epitope area of the parent protein by genetic engineering mutations of a DNA sequence encoding the parent protein,

(e) introducing the mutated DNA sequence into a suitable host, culturing the host and expressing the protein variant,

(f) evaluating the immunogenicity of the protein variant using the parent protein as reference,

(g) introducing the mutated DNA sequence into an expression construct and transforming a suitable plant cell with the construct, and

(h) regenerating the plant from the plant cell.

In a second aspect the invention relates to a transgenic plant transformed with a nucleotide sequence encoding a protein allergen having modified immunogenicity as compared to a parent protein.

Another aspect is a DNA molecule encoding a protein variant as defined above.

A further aspect is a vector comprising a DNA molecule as described above as well a host cell comprising said DNA molecule.

### DEFINITIONS

Production of Low-allergenic Proteins

Prior to a discussion of the detailed embodiments of the invention, a definition of specific terms related to the main aspects of the invention is provided.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II /D.N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984), Methods in Plant Mol. Biol. And Biotechnology, (Glick B. & Thompson J. (eds.) CRC Press Inc., Boca Raton, Fla.), Plant Molecular Biology Manual A6, Klywer Academic Publisher, Dordrecht, The Netherlands.

When applied to a protein, the term “isolated” indicates that the protein is found in a condition other than its native environment. In a preferred form, the isolated protein is substantially free of other proteins. It is preferred to provide the proteins in a highly purified form, i.e., greater than 95% pure, more preferably greater than 99% pure. When applied to a polynucleotide molecule, the term “isolated” indicates that the molecule is removed from its natural genetic milieu, and is thus free of other extraneous or unwanted coding sequences, and is in a form suitable for use within genetically engineered protein production systems. Such isolated molecules are those that are separated from their natural environment and include cDNA and genomic DNA clones. Isolated DNA molecules of the present invention are free of other genes with which they are ordinarily associated, and may include naturally occurring 5′ and 3′ untranslated regions such as promoters and terminators. The identification of associated regions will be evident to one of ordinary skill in the art (see for example, Dynan and Tijan, Nature 316: 774-78, 1985).

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A DNA “coding sequence” is a double-stranded DNA sequence, which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

A coding sequence is “under the control” of transcriptional and translational control sequences in a cell when RNA polymerase transcribes the coding sequence into mRNA, which is then trans-RNA spliced and translated into the protein encoded by the coding sequence.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

“Operably linked”, when referring to DNA segments, indicates that the segments are arranged so that they function in concert for their intended purposes, e.g. transcription initiates in the promoter and proceeds through the coding segment to the terminator.

“Heterologous” DNA refers to DNA not naturally located in the cell, or in a chromosomal site of the cell. Preferably, the heterologous DNA includes a gene foreign to the cell.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

A “clone” is a population of cells derived from a single cell or common ancestor by mitosis.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Nucleic Acid Sequence:

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain producing the polypeptide, or from another related organism and thus, for example, may be an allelic or species variant of the polypeptide encoding region of the nucleic acid sequence.

Nucleic Acid Construct:

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a polypeptide of interest. The construct may optionally contain other nucleic acid segments.

The DNA of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleic acid construct may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

The term nucleic acid construct may be synonymous with the term expression cassette when the nucleic acid construct contains all the control sequences required for expression of a coding sequence of the present invention.

The term “control sequences” is defined herein to include all components which are necessary or advantageous for expression of the coding sequence of the nucleic acid sequence. Each control sequence may be native or foreign to the nucleic acid sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

The control sequence may be an appropriate promoter sequence, a nucleic acid sequence which is recognized by a host cell for expression of the nucleic acid sequence. The promoter sequence contains transcription and translation control sequences which mediate the expression of the polypeptide. The promoter may be any nucleic acid sequence which shows transcriptional activity in the host cell of choice and may be obtained from genes encoding extracellular or intracellular polypeptides either homologous or heterologous to the host cell.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region, which codes for an amino acid sequence linked to the amino terminus of the polypeptide which can direct the expressed polypeptide into the cell's secretory pathway of the host cell. The 5′ end of the coding sequence of the nucleic acid sequence may inherently contain a signal peptide coding region naturally linked in translation reading frame with the segment of the coding region which encodes the secreted polypeptide.

Alternatively, the 5′ end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

The control sequence may also be a propeptide coding region, which codes for an amino acid sequence positioned at the amino terminus of a polypeptide. The resultant polypeptide is known as a pro-enzyme or pro-polypeptide (or a zymogen in some cases). A pro-polypeptide is generally inactive and can be converted to mature active polypeptide by catalytic or autocatalytic cleavage of the propeptide from the pro-polypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

The nucleic acid constructs of the present invention may also comprise one or more nucleic acid sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleic acids encoding one or more of these factors are not necessarily in tandem with the nucleic acid sequence encoding the polypeptide.

An activator is a protein which activates transcription of a nucleic acid sequence encoding a polypeptide (Kudla et al., 1990, EMBO Journal 9:1355-1364; Jarai and Buxton, 1994, Current Genetics 26:2238-244; Verdier, 1990, Yeast 6:271-297). The nucleic acid sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra and MacKenzie et al., 1993, Journal of General Microbiology 139:2295-2307.

A chaperone is a protein which assists another polypeptide in folding properly (Hartl et al., 1994, TIBS 19:20-25; Bergeron et al., 1994, TIBS 19:124-128; Demolder et al., 1994, Journal of Biotechnology 32:179-189; Craig, 1993, Science 260:1902-1903; Gething and Sambrook, 1992, Nature 355:33-45; Puig and Gilbert, 1994, Journal of Biological Chemistry 269:7764-7771; Wang and Tsou, 1993, The FASEB Journal 7:1515-11157; Robinson et al., 1994, Bio/Technology 1:381-384). The nucleic acid sequence encoding a chaperone may be obtained from the genes encoding Bacillus subtilis GroE proteins, Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, 1992, supra, and Hartl et al., 1994, supra.

A processing protease is a protease that cleaves a propeptide to generate a mature biochemically active polypeptide (Enderlin and Ogrydziak, 1994, Yeast 10:67-79; Fuller et al., 1989, Proceedings of the National Academy of Sciences USA 86:1434-1438; Julius et al., 1984, Cell 37:1075-1089; Julius et al., 1983, Cell 32:839-852). The nucleic acid sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xpr6).

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GAL1 system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be placed in tandem with the regulatory sequence.

Promoters

Examples of suitable promoters for directing the transcription of the nucleic acid constructs of the present invention, especially in a bacterial host cell, are the promoters obtained from the E. coli lac operon, the Streptomyces coelicolor agarase gene (dagA), the Bacillus subtilis levan sucrase gene (sacB), the Bacillus subtilis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75:3727-3731), as well as the tac promoter (DeBoer et al., 1983, Proceedings of the National Academy of Sciences USA 80:21-25), or the Bacillus pumilus xylosidase gene, or by the phage Lambda PR or PL promoters or the E. coli lac, trp or tac promoters. Further promoters are described in “Useful proteins from recombinant bacteria” in Scientific American, 1980, 242:74-94; and in Sambrook et al., 1989, supra.

Examples of suitable promoters for directing the transcription of the nucleic acid constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (-amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for directing the transcription of the DNA encoding the polypeptide of the invention in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. Nos. 5,155,037; 5,162,222), or the baculovirus 39K delayed-early gene promoter (U.S. Pat. Nos. 5,155,037; 5,162,222).

Terminators

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. for fungal hosts) the TPI1 (Alber and Kawasaki, op. cit.) or ADH3 (McKnight et al., op. cit.) terminators.

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, supra.

Polyadenylation Signals

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Useful polyadenylation sequences for yeast host cells are described by Guo and Sherman, 1995, Molecular Cellular Biology 15:5983-5990.

Polyadenylation sequences are well known in the art for mammalian host cells such as SV40 or the adenovirus 5 Elb region.

Signal Sequences

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57:109-137.

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase or lipase gene, or the Rhizomucor miehei lipase or protease gene, Aspergillus sp. amylase or glucoamylase, a gene encoding a Rhizomucor miehei lipase or protease. The signal peptide is preferably derived from a gene encoding A. oryzae TAKA amylase, A. niger neutral (-amylase, A. niger acid-stable amylase, or A. niger glucoamylase.

Useful signal peptides for yeast host cells are obtained from the genes for Saccharomyces cerevisiae a-factor and Saccharomyces cerevisiae invertase. Other useful signal peptide coding regions are described by Romanos et al., 1992, supra.

For secretion from yeast cells, the secretory signal sequence may encode any signal peptide which ensures efficient direction of the expressed polypeptide into the secretory pathway of the cell. The signal peptide may be naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the polypeptide. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

Expression Vectors

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression, and possibly secretion.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol, tetracycline, neomycin, hygromycin or methotrexate resistance. A frequently used mammalian marker is the dihydrofolate reductase gene (DHFR). Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleic acid sequence encoding the polypeptide or any other element of the vector for stable integration of the vector into the genome by homologous or non-homologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination. These nucleic acid sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMβ1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

More than one copy of a nucleic acid sequence encoding a polypeptide of the present invention may be inserted into the host cell to amplify expression of the nucleic acid sequence. Stable amplification of the nucleic acid sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a nucleic acid sequence of the invention, which are advantageously used in the recombinant production of the polypeptides. The term “host cell” encompasses any progeny of a parent cell which is not identical to the parent cell due to mutations that occur during replication.

The cell is preferably transformed with a vector comprising a nucleic acid sequence of the invention followed by integration of the vector into the host chromosome. “Transformation” means introducing a vector comprising a nucleic acid sequence of the present invention into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination as described above.

The choice of a host cell will to a large extent depend upon the gene encoding the polypeptide and its source. The host cell may be from a unicellular microorganism, e.g., a prokaryote, or from a non-unicellular microorganism, e.g., a eukaryote.

Non-glycosylating Host Cells

Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

Glycosylating Host Cells

The host cell may be a eukaryote, such as a mammalian cell, an insect cell, a plant cell or a fungal cell. Useful mammalian cells include Chinese hamster ovary (CHO) cells, HeLa cells, baby hamster kidney (BHK) cells, COS cells, or any number of other immortalized cell lines available, e.g., from the American Type Culture Collection.

Examples of suitable mammalian cell lines are the COS (ATCC CRL 1650 and 1651), BHK (ATCC CRL 1632, 10314 and 1573, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds., Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

The yeast host cell may be selected from a cell of a species of Candida, Kluyveromyces, Saccharomyces, Schizosaccharomyces, Candida, Pichia, Hansenula or Yarrowia. In a preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis, Kluyveromyces fragilis, Hansenula polymorpha, Pichia pastoris Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. Nos. 4,882,279 and 4,879,231).

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger, Aspergillus nidulans or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another prefered embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell or a Acremonium chrysogenum cell. In another most preferred embodiment, the Trichoderma cell is a Trichoderma harzianum, Trichoderma koningii, Trichoderma longibrachiatum, Trichoderma reesei or Trichoderma viride cell. The use of Aspergillus spp. for the expression of proteins is described in, e.g., EP 272 277, EP 230 023.

Transformation

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81:1470-1474. A suitable method of transforming Fusarium species is described by Malardier et al., 1989, Gene 78:147-156 or in copending U.S. Ser. No. 08/269,449. Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78: 147-156.

Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920. Mammalian cells may be transformed by direct uptake using the calcium phosphate precipitation method of Graham and Van der Eb (1978, Virology 52:546).

Transformation of insect cells and production of heterologous polypeptides therein may be performed as described in U.S. Pat. Nos. 4,745,051; 4,775,624; 4,879,236; 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

Methods of Production

The transformed or transfected host cells described above are cultured in a suitable nutrient medium under conditions permitting the production of the desired molecules, after which these are recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

If the molecules are secreted into the nutrient medium, they can be recovered directly from the medium. If they are not secreted, they can be recovered from cell lysates. The molecules are recovered from the culture medium by conventional procedures including separating the host cells from the medium by centrifugation or filtration, precipitating the proteinaceous components of the supernatant or filtrate by means of a salt, e.g. ammonium sulphate. The molecules of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J-C Janson and Lars Ryden, editors, VCH Publishers, New York, 1989).

The molecules of interest may be detected using methods known in the art that are specific for the molecules. These detection methods may include use of specific antibodies, formation of a product, or disappearance of a substrate. For example, an enzyme assay may be used to determine the activity of the molecule. Procedures for determining various kinds of activity are known in the art.

Production of Transgenic Plants

Cloning a DNA Sequence Encoding a Modified Protein

The nucleotide sequence encoding the protein of the invention may be of any origin, including mammalian, plant and microbial origin and may be isolated from these sources by conventional methods.

The DNA sequence encoding a parent protein may be isolated from the cell producing the protein in question, using various methods well known in the art. First, a genomic DNA and/or cDNA library should be constructed using chromosomal DNA or messenger RNA from the organism that produces the protein to be studied. Then, if the amino acid sequence of the protein is known, homologous, labelled oligonucleotide probes may be synthesised and used to identify protein-encoding clones from a genomic library prepared from the organism in question. Alternatively, a labelled oligonucleotide probe containing sequences homologous to a known protein gene could be used as a probe to identify protein-encoding clones, using hybridization and washing conditions of lower stringency.

Alternatively, the DNA sequence encoding the protein may be prepared synthetically by established standard methods, e.g. the phosphoroamidite method described by S. L. Beaucage and M. H. Caruthers (1981) or the method described by Matthes et al. (1984). In the phosphoroamidite method, oligonucleotides are synthesized, e.g. in an automatic DNA synthesizer, purified, annealed, ligated and cloned in appropriate vectors.

Finally, the DNA sequence may be of mixed genomic and synthetic origin, mixed synthetic and cDNA origin or mixed genomic and cDNA origin, prepared by ligating fragments of synthetic, genomic or cDNA origin, wherein the fragments correspond to various parts of the entire DNA sequence, in accordance with techniques well known in the art. The DNA sequence may also be prepared by polymerase chain reaction (PCR) using specific primers, for instance as described in U.S. Pat. No. 4,683,202 or R. K. Saiki et al. (1988). See also WO 99/43794 disclosing how to make variants, e.g. by use of mutagenesis techniques known in the art.

Expression Constructs

In order to accomplish expression of the protein in seeds of the transgenic plant of the invention the nucleotide sequence encoding the protein is inserted into an expression construct containing regulatory elements capable of directing the expression of the nucleotide sequence and, if necessary, to direct secretion of the gene product or targeting of the gene product to the seeds of the plant. Manipulation of nucleotide sequences using restriction endonucleases to cleave DNA molecules into fragments and DNA ligase enzymes to unite compatible fragments into a single DNA molecule with subsequent incorporation into a suitable plasmid, cosmid, or other transformation vector are well known in the art.

In order for transcription to occur the nucleotide sequence encoding the protein is operably linked to a suitable promoter capable of mediating transcription in the plant in question. The promoter may be an inducible promoter or a constitutive promoter. Typically, an inducible promoter mediates transcription in a tissue-specific or growth-stage specific manner, whereas a constitutive promoter provides for sustained transcription in all cell tissues. An example of a suitable constitutive promoter useful for the present invention is the cauliflower mosaic virus 35 S promoter. Other constitutive promoters are transcription initiation sequences from the tumor-inducing plasmid (Ti) of Agrobacterium such as the octopine synthase, nopaline synthase, or mannopine synthase initiator.

Examples of suitable inducible promoters include a seed-specific promoter, a promoter of the gene encoding a rice seed storage protein such as glutelin, prolamin, globulin or albumin (Wu et al., Plant and Cell Physiology Vol. 39, No. 8 pp. 885-889 (1998)), a Vicia faba promoter from the legumin B4 and the unknown seed protein gene from Vicia faba described by Conrad U. et al, Journal of Plant Physiology Vol. 152, No. 6 pp. 708-711 (1998), the storage protein napA promoter from Brassica napus, or any other seed specific promoter known in the art, e.g. as described in WO 91/14772.

In order to increase the expression of the protein it is desirable that a promoter enhancer element is used. For instance, the promoter enhancer may be an intron which is placed between the promoter and the amylase gene. The intron may be one derived from a monocot or a dicot. For instance, the intron may be the first intron from the rice Waxy (Wx) gene (Li et al., Plant Science Vol. 108, No. 2, pp. 181-190 (1995)), the first intron from the maize Ubi1 (Ubiquitin) gene (Vain et al., Plant Cell Reports Vol. 15, No. 7 pp. 489-494 (1996)) or the first intron from the Act1 (actin) gene. As an example of a dicot intron the chsA intron (Vain et al. op cit.) is mentioned. Also, a seed specific enhancer may be used to increase the expression of the protein in seeds. An example of a seed specific enhancer is the one derived from the beta-phaseolin gene encoding the major seed storage protein of bean (Phaseolus vulgaris) disclosed by Vandergeest and Hall, Plant Molecular Biology Vol. 32, No. 4, pp. 579-588 (1996).

Also, the expression construct contains a terminator sequence to signal transcription termination of the protein gene such as the rbcS2′ and the nos3′ terminators.

To facilitate selection of successfully transformed plants, the expression construct should also include one or more selectable markers, e.g. an antibiotic resistance selection marker or a selection marker providing resistance to a herbicide. One widely used selection marker is the neomycin phosphotransferase gene (NPTII) which provides kanamycin resistance. Examples of other suitable markers include a marker providing a measurable enzyme activity, e.g. dihydrofolate reductase, luciferase, and β-glucoronidase (GUS). Phosphinothricin acetyl transferase may be used as a selection marker in combination with the herbicide basta or bialaphos.

Transgenic Plant Species

In the present context the term “transgenic plant” is intended to mean a plant which has been genetically modified to express a protein of interest and progeny of such plant having retained the capability of producing a the protein. The term also includes a part of such plant such as a leaf, seed, stem, any tissue from the plant, an organelle, a cell of the plant, etc.

Any transformable seed-producing plant species may be used for the present invention. Of particular interest is a monocotyledonous plant species, in particular crop or cereal plants such as wheat (Triticum, e.g. aestivum), barley (Hordeum, e.g. vulgare), oats, rye, rice, sorghum and corn (Zea, eg mays). In particular, wheat is preferred.

Transformation of Plants

The transgenic plant cell of the invention may be prepared by methods known in the art. The transformation method used will depend on the plant species to be transformed and can be selected from any of the transformation methods known in the art such as Agrobacterium mediated transformation (Zambryski et al., EMBO Journal 2, pp 2143-2150, 1993), particle bombardment (Vasil et al. 1991), electroporation (Fromm et al. 1986, Nature 319, pp 791-793), and virus mediated transformation. For transformation of monocots particle bombardment (i.e. biolistic transformation) of embryogenic cell lines or cultured embryos are preferred. In the following references disclosing methods for transforming different plants are mentioned together with the plant: Rice (Cristou et al. 1991, Bio/Technology 9, pp. 957-962), Maize (Gordon-Kamm et al. 1990, Plant Cell 2, pp. 603-618), Oat (Somers et al. 1992, Bio/Technology 10, pp 1589-1594), Wheat (Vasil et al. 1991, Bio/Technology 10, pp. 667-674, Weeks et al. 1993, Plant Physiology 102, pp. 1077-1084) and barley (Wan and Lemaux 1994, Plant Physiology 102, pp. 37-48, review Vasil 1994, Plant Mol. Biol. 25, pp 925-937).

More specifically, Agrobacterium mediated transformation is conveniently achieved as follows:

A vector system carrying the protein is constructed. The vector system may comprise one vector, but it can comprise two vectors. In the case of two vectors the vector system is referred to as a binary vector system (Gynheung An et al.(1980), Binary Vectors, Plant Molecular Biology Manual A3, 1-19).

An Agrobacterium based plant transformation vector consists of replication origin(s) for both E. coli and Agrobacterium and a bacterial selection marker. A right and preferably also a left border from the Ti plasmid from Agrobacterium tumefaciens or from the Ri plasmid from Agrobacterium rhizogens is nessesary for the transformation of the plant. Between the borders the expression construct is placed which contains the protein gene and appropriate regulatory sequences such as promotor and terminator sequences. Additionally, a selection gene e.g. the neomycin phosphotransferase type II (NPTII) gene from transposon Tn5 and a reporter gene such as the GUS (betha-glucuronidase) gene is cloned between the borders. A disarmed Agrobacterium strain harboring a helper plasmid containing the virulens genes is transformed with the above vector. The transformed Agrobacterium strain is then used for plant transformation.

Immunological Definitions

The term “immunological response”, used in connection with the present invention, is the response of an organism to a compound, which involves the immune system according to any of the four standard reactions (Type I, II, III and IV according to Coombs & Gell).

Correspondingly, the “immunogenicity” of a compound used in connection with the present invention refers to the ability of this compound to induce an ‘immunological response’ in animals including man.

The term “allergic response”, used in connection with the present invention, is the response of an organism to a compound, which involves IgE mediated responses (Type I reaction according to Coombs & Gell). It is to be understood that sensibilization (i.e. development of compound-specific IgE antibodies) upon exposure to the compound is included in the definition of “allergic response”.

Correspondingly, the “allergenicity” of a compound used in connection with the present invention refers to the ability of this compound to induce an ‘allergic response’ in animals including man.

The term “parent protein” refer to the polypeptide to be modified by creating a library of diversified mutants. The “parent protein” may be a naturally occurring (or wild-type) polypeptide or it may be a variant thereof prepared by any suitable means. For instance, the “parent protein” may be a variant of a naturally occurring polypeptide which has been modified by substitution, deletion or truncation of one or more amino acid residues or by addition or insertion of one or more amino acid residues to the amino acid sequence of a naturally-occurring polypeptide.

The term “randomized library” of protein variants refers to a library with at least partially randomized composition of the members, e.g. protein variants.

An “epitope” is a set of amino acids on a protein that are involved in an immunological response, such as antibody binding or T-cell activation. One particularly useful method of identifying epitopes involved in antibody binding is to screen a library of peptide-phage membrane protein fusions and selecting those that bind to relevant antigen-specific antibodies, sequencing the randomized part of the fusion gene, aligning the sequences involved in binding, defining consensus sequences based on these alignments, and mapping these consensus sequences on the surface or the sequence and/or structure of the antigen, to identify epitopes involved in antibody binding.

By the term “epitope pattern” is meant such a consensus sequence of antibody binding peptides. An example is the epitope pattern A R R<R (SEQ ID NO: 2). The sign “<” in this notation indicates that the aligned antibody binding peptides included a non-consensus amino acid between the second and the third arginine.

An “epitope area” is defined as the amino acids situated close to the epitope sequence amino acids. Preferably, the amino acids of an epitope area are located <5 Å from the epitope sequence. Hence, an epitope area also includes the corresponding epitope sequence itself. Modifications of amino acids of the ‘epitope area’ can possibly affect the immunogenic function of the corresponding epitope.

By the term “epitope sequence” is meant the amino acid residues of a parent protein, which have been identified to belong to an epitope by the methods of the present invention (an example of an epitope sequence is E271 Q12 I8 in Savinase).

The term ‘antibody binding peptide’ denotes a peptide that bind with sufficiently high affinity to antibodies. Identification of ‘antibody binding peptides’ and their sequences constitute the first step of the method of this invention.

“Anchor amino acids” are the individual amino acids of an epitope pattern.

“Hot spot amino acids” are amino acids of parent protein, which are particularly likely to result in modified immunogenicity if they are mutated. Amino acids, which appear in three or more epitope sequences or which correspond to anchor amino acids are hot spot amino acids.

“Environmental allergens” are protein allergens that are present naturally. They include pollen, dust mite allergens, pet allergens, food allergens, venoms, etc.

“Commercial allergens” are protein allergens that are being brought to the market commercially. They include enzymes, pharmaceutical proteins, antimicrobial peptides, as well as allergens of transgenic plants.

The “donor protein” is the protein that was used to raise antibodies used to identify antibody binding sequences, hence the donor protein provides the information that leads to the epitope patterns.

The “acceptor protein” is the protein, whose structure is used to fit the identified epitope patterns and/or to fit the antibody binding sequences. Hence the acceptor protein is also the parent protein.

An “autoepitope” is one that has been identified using antibodies raised against the parent protein, i.e. the acceptor and the donor proteins are identical.

A “heteroepitope” is one that has been identified with distinct donor and acceptor proteins.

The term “functionality” of protein variants refers to e.g. enzymatic activity; binding to a ligand or receptor; stimulation of a cellular response (e.g. ³H-thymidine incorporation as response to a mitogenic factor); or anti-microbial activity.

By the term “specific polyclonal antibodies” is meant polyclonal antibodies isolated according to their specificity for a certain antigen, e.g. the protein backbone.

By the term “monospecific antibodies” is meant polyclonal antibodies isolated according to their specificity for a certain epitope. Such monospecific antibodies will bind to the same epitope, but with different affinity, as they are produced by a number of antibody producing cells recognizing overlapping but not necessarily identical epitopes.

‘Spiked mutagenesis’ is a form of site-directed mutagenesis, in which the primers used have been synthesized using mixtures of oligonucleotides at one or more positions.

By the term “a protein variant having modified immunogenicity as compared to the parent protein” is meant a protein variant which differs from the parent protein in one or more amino acids whereby the immunogenicity of the variant is modified. The modification of immunogenicity may be confirmed by testing the ability of the protein variant to elicit an IgE/IgG response.

In the present context the term “protein” is intended to cover oligopeptides, polypeptides as well as proteins as such.

### DETAILED DESCRIPTION OF THE INVENTION

The present invention relates to a method of producing a plant expressing a protein variant having modified immunogenicity as compared to a parent protein, comprising the steps of:

(a) obtaining antibody binding peptide sequences involved in antibody binding,

(b) using the sequences to localize epitope sequences on the primary and/or the 3-dimensional structure of a parent protein,

(c) defining an epitope area including amino acids situated within 5 Å from the epitope amino acids constituting the epitope sequence,

(d) changing one or more of the amino acids defining the epitope area of the parent protein by genetic engineering mutations of a DNA sequence encoding the parent protein,

(e) introducing the mutated DNA sequence into a suitable host, culturing the host and expressing the protein variant,

(f) evaluating the immunogenicity of the protein variant using the parent protein as reference,

(g) introducing the mutated DNA sequence into an expression construct and transforming a suitable plant cell with the construct, and

(h) regenerating the plant from the plant cell.

Allergens

Many allergens are known that elicit allergic responses, which may range is severity from mildly irritating to life-threatening.

Food allergies are mediated through the interaction of IgE to specific proteins contained within the food. Examples of common food allergens include proteins from peanuts, milk, grains such as wheat and barley, soybeans, eggs, fish, crustaceans, and molluscs. These account for greater than 90% of the food allergies (Taylor, Food Techn. 39, 146-152 (1992). The IgE binding epitopes from the major allergens of cow milk (Ball, et al. (1994) Clin. Exp. Allergy, 24, 758-764), egg (Cooke, S. K. and Sampson, H. R. (1997) J. Immunol., 159, 2026-2032), codfish (Aas, K., and Elsayed, S. (1975) Dev. Biol. Stand. 29, 90-98), hazel nut (Elsayed, et al. (1989) Int. Arch. Allergy Appl. Immunol. 89, 410-415), peanut (Burks et al. (1997) Eur. J. Biochemistry, 245:334-339; Stanley et al. (1997) Archives of Biochemistry and Biophysics, 342:244-253), soybean (Herein et al. (1990) Int. Arch. Allergy Appl. Immunol. 92, 193-198) and scrimp (Shanty et al. (1993) J. Immunol. 151, 5354-5363) have all been elucidated as have others.

Cross-reactivity of allergens occurs if different proteins are more or less homologous and contain identical or nearly identical epitopes. Frequently, it can be classified and explained on the basis of taxonomic relationships, because closely related organisms often have great similarities and share a number of antigens, e.g. pollen from different species of the same genus/family. It should be noted however, that cross-reactions also may be caused by evolutionary conserved protein structures. Profilin, a conserved protein in eukaryotic cells, is responsible for most of the cross-reactivity between birch pollen allergen and extracts of vegetables. The consequence of a strong cross-reactivity is the sensitization to allergens without exposure (see Mohapatra (1993) In: Kraft D, Sehon A (eds) Molecular Biology and Immunology of Allergens. Boca Raton, Ann Arbor, London, Tokyo: CRC Press: 69-81 and Akkerdaas, et al (1995) Allergy 50: 215-220).

A related objective is to reduce the allergenicity of food proteins and plants producing these proteins to reduce cross-reactivity between food allergens and other environmental allergens and cross-reactivity between food allergens and commercial allergens. Cross-reactivities between environmental allergens (like pollen, dust mites etc.) and commercial allergens (like enzyme proteins) have been established in the literature (J. All. Clin. Immunol., 1998, vol. 102, pp. 679-686 and by the present inventors. The molecular reason for this cross-reactivity can be explored using epitope mapping. By finding epitope patterns using antibodies raised against a commercial allergen (donor protein) and mapping this information on a environmental allergen (the acceptor protein), one may find the epitopes that are common to both proteins, and hence responsible for the cross-reactivity.

Testing of this approach would be done using an antibody-binding assay with the protein variant (and its parent protein as control) and antibodies raised against the protein that cross-reacts with the parent protein. The method is otherwise identical to those described in the Methods section for characterization of allergencitiy and antigenicity.

Pollen allergens include but are not limited to those of the order Fagales, Oleales, Pinales, Poales, Asterales, and Urticales; including those from Betula, Alnus, Corylus, Carpinus, Olea, Phleum pratense and Artemisia vulgaris, such as Aln g1, Cor a1, Car b1, Cry j1, Amb a1 and a2, Art v1, Par j1, Ole e1, Ave v1, and Bet v1 (WO 99/47680).

Other allergens include proteins from insects such as flea, tick, mite, fire ant, cockroach, and bee as well as molds, dust, grasses, trees, weeds, fungi, venom and proteins from mammals including horses, dogs, cats, etc.

Mite allergens include but are not limited to those from Derm. farinae and Derm. pteronys, such as Der f1 and f2, and Der p1 and p2.

From mammals, relevant environmental allergens include but are not limited to those from cat, dog, and horse as well as from dandruff from the hair of those animals, such as Fel d1, Can f1 Equ c1, c2, c3.

Venum allergens include but are not limited to PLA2 from bee venom as well as Apis m1 and m2, Ves g1, g2 and g5, and te Pol and Sol allergens.

Fungal allergens include those from Alternaria alt. and Cladospo. herb. such as Alt a1 and Cla h1.

Latex products are manufactures from a milky fluid derived from the rubber tree Hevea brasiliensis and other processing chemicals. A number of the proteins in latex can cause a range of allergic reactions. Many products contain latex, such as medical supplies and personal protective equipment. Three types of reactions can occur in persons sensitive to latex: Irritant contact dermatitis, and immediate systemic hypersensitivity. Additionally, the proteins responsible for the allergic reactions can fasten to the powder of latex gloves. This powder can be inhaled, causing exposure through the lungs. Proteins found in latex that interact with IgE antibodies were characterized by two-dimensional electrophoresis. Protein fractions of 56, 45, 30, 20, 14, and less than 6.5 kD were detected (Posch A. et al., (1997) J. Allergy Clin. Immunol. 99(3), 386-395). Acidic proteins in the 8-14 kD and 22-24 kD range that reacted with IgE antibodies were also identified (Posch A. et al. (1997) J. Allergy Clin. Immunol. 99(3), 385-395. The proteins prohevein and hevein, from hevea brasiliensis, are known to be major latex allergens and to interact with IgE (Alenius, H. et al., Clin. Exp. Allergy 25(7), 659-665; Chen Z. et al., (1997) J. Allergy Clin. Immunol. 99(3), 402-409). Most of the IgE binding domains have been shown to be in the hevein domain rather than the domain specific for prohevein (Chen Z. et al., (1997) J. Allergy Cclin. Immunol. 99(3), 402-409). The main IgE binding epitope of prohevein is thought to be in the N-terminal, 43 aamino acid fragment (Alenius H. et al. (1996) J. Immunol. 156(4), 1618-1625). The hevein lectin family of proteins has been shown to have homology with potato lectin and snake venom disintegrins (platelet aggregation inhibitors) (Kielisqewski, M. L. et al. (1994) Plant J. 5(6), 849-861).

A number of proteins of interest for expression in transgenic plants could be useful objects for epitope engineering. If for instance a heterologous enzyme is introduced into a transgenic plant e.g. to increase the nutritional value of food or feed derived from that plant, that enzyme may lead to allergenicity problems in humans or animals ingesting the plant-derived material. Epitope mapping and engineering of such heterologous enzymes or other proteins of transgenic plants may lead to reduction or elimination of this problem. Hence, the methods of this patent are also useful for potentially modifying proteins for heterologous expression in plants and plant cells.

a) How to Find Antibody Binding Peptide Sequences and Epitope Patterns

A first step of the method is to identify peptide sequences, which bind specifically to antibodies.

Antibody binding peptide sequences can be found by testing a set of known peptide sequences for binding to antibodies raised against the donor protein, e.g. by using pooled sera from allergic patients. These sequences are typically selected, such that each represents a segment of the donor protein sequence (Mol. Immunol., 1992, vol. 29, pp.1383-1389; Am. J. Resp. Cell. Mol. Biol. 2000, vol. 22, pp. 344-351). Also, randomized synthetic peptide libraries can be used to find antibody binding sequences (Slootstra et al; Molecular Diversity, 1996, vol. 2, pp. 156-164).

In a preferred method, the identification of antibody binding sequences may be achieved by screening of a display package library, preferably a phage display library. The principle behind phage display is that a heterologous DNA sequence can be inserted in the gene coding for a coat protein of the phage (WO 92/15679). The phage will make and display the hybrid protein on its surface where it can interact with specific target agents. Such target agent may be antigen-specific antibodies. It is therefore possible to select specific phages that display antibody-binding peptide sequences. The displayed peptides can be of predetermined lengths with randomized sequences, resulting in a random peptide display package library. Thus, by screening for antibody binding, one can isolate the peptide sequences that have sufficiently high affinity for the particular antibody used. The peptides of the hybrid proteins of the specific phages which bind protein-specific antibodies characterize epitopes that are recognized by the immune system.

The antibodies used for reacting with the display package are preferably IgE antibodies to ensure that the epitopes identified are IgE epitopes, i.e. epitopes inducing and binding IgE. In a preferred embodiment the antibodies are polyclonal antibodies, optionally monospecific antibodies.

For the purpose of the present invention polyclonal antibodies are preferred in order to obtain a broader knowledge about the epitopes of a protein.

It is of great importance that the amino acid sequence of the peptides presented by the display packages is long enough to represent a significant part of the epitope to be identified. In a preferred embodiment of the invention the peptides of the peptide display package library are oligopeptides having from 5 to 25 amino acids, preferably at least 8 amino acids, such as 9 amino acids. For a given length of peptide sequences (n), the theoretical number of different possible sequences can be calculated as 20ⁿ. The diversity of the package library used must be large enough to provide a suitable representation of the theoretical number of different sequences. In a phage-display library, each phage has one specific sequence of a determined length. Hence an average phage display library can express 10⁸-10¹² different random sequences, and is therefore well-suited to represent the theoretical number of different sequences.

The antibody binding peptide sequences can be further analysed by consensus alignment e.g. by the methods described by Feng and Doolittle, Meth. Enzymol., 1996, vol. 266, pp. 368-382; Feng and Doolittle, J. Mol. Evol., 1987, vol. 25, pp. 351-360; and Taylor,. Meth. Enzymol., 1996, vol. 266, pp. 343-367.

This leads to identification of epitope patterns, which can assist the comparison of the linear information obtained from the antibody binding peptide sequences to the 3-dimensional structure of the acceptor protein in order to identify epitope sequences at the surface of the acceptor protein.

b) How to Identify Epitope Sequences and Epitope Areas

Given a number of antibody binding peptide sequences and possibly the corresponding epitope patterns, one need the 3-dimensional structure coordinates of an acceptor protein to find the epitope sequences on its surface.

These coordinates can be found in databases (NCBI: http://www.ncbi.nlm.nih.gov/), determined experimentally using conventional methods (Ducruix and Giegé: Crystallization of Nucleic Acids and Proteins, IRL PRess, Oxford, 1992, ISBN 0-19-963245-6), or they can be deduced from the coordinates of a homologous protein. Typical actions required for the construction of a model structure are: alignment of homologous sequences for which 3-dimensional structures exist, definition of Structurally Conserved Regions (SCRs), assignment of coordinates to SCRs, search for structural fragments/loops in structure databases to replace Variable Regions, assignment of coordinates to these regions, and structural refinement by energy minimization. Regions containing large inserts (>3 residues) relative to the known 3-dimensional structures are known to be quite difficult to model, and structural predictions must be considered with care.

Using the coordinates and the several methods of mapping the linear information on the 3-dimensional surface are possible, as described in the examples below.

One can match each amino acid residue of the antibody binding peptide to an identical or homologous amino acid on the 3-D surface of the acceptor protein, such that amino acids that are adjacent in the primary sequence are close on the surface of the acceptor protein, with close being <5 Å, preferably <3 Å between any two atoms of the two amino acids.

Alternatively, one can define a geometric body (e.g. an ellipsoid, a sphere, or a box) of a size that matches a possible binding interface between antibody and antigen and look for a positioning of this body where it will contain most of or all the anchor amino acids.

The anchor amino acid residues are transferred to a three dimensional structure of the protein of interest, by colouring D red, F white and K blue. Any surface area having all three residues within a distance of 18 Å, preferably 15 Å, more preferably 12 Å, is then claimed to be an epitope. The relevant distance can easily be measured using e.g. molecular graphics programs like InsightII from Molecular Simulations Inc.

Also, one can use the epitope patterns to facilitate identification of epitope sequences. This can be done, by first matching the anchor amino acids on the 3-D structure and subsequently looking for other elements of the antibody binding peptide sequences, which provide additional matches. If there are many residues to be matched, it is only necessary that a suitable number can be found on the 3-D structure. For example if an epitope pattern comprises 4, 5, 6, or 7 amino acids, it is only necessary that 3 matches surface elements of the acceptor protein.

In all cases, it is desirable that amino acids of the epitope sequence are surface exposed (as described below in Examples).

It is known, that amino acids that surround binding sequences can affect binding of a ligand without participating actively in the binding process. Based on this knowledge, areas covered by amino acids with potential steric effects on the epitope-antibody interaction, were defined around the identified epitope sequences. These areas are called ‘epitope areas’. Practically, all amino acids situated within 5 Å from the amino acids defining the epitope sequence were included. Preferably, the epitope area equals the epitope sequence. The accessibility criterium was not used as hidden amino acids of an epitope area also can have an effect on the adjacent amino acids of the epitope sequence.

In case the 3D structure of the target protein is not available, an alternative method is used for the identification of the overall area involved in antibody binding. This method is called here ‘vitual screening’, and is based upon sequence alignment. Sequences are known for most environmental allergens (Liebers et al (1996) Clin Exp Allergy 26: 494-516).

Two approaches can be distinguished.

(a) Given a target protein with known sequence that cross-reacts with a number of well-characterized allergens with known sequence and partial homology with the target protein, sequence alignment will identify the homologous stretches that might be involved in cross-reactive antibody binding. This approach is applicable on most environmental allergens, as extensive reports on cross-reactions between these allergens exist.

(b) Given a target protein with known sequence that does not cross-react with one or several proteins that are >60% homologous, sequence alignment will identify the areas that are different and thus might be involved in antibody binding.

Eventually, either approach can be combined with 3D structure building using e.g. proteins with functional similarities as starting point.

In both cases (A and B), the identified areas might be subjected to protein engineering.

c) How to Use the Epitope Information

There are several ways to utilize the information about epitope sequences, which has been derived by the methods of this invention: Reduce the allergenicity of an allergen using protein engineering; reduce the potential of commercial proteins to cross-react with environmental allergens and hence cause allergic reactions in people sensitized to the environmental allergens (information about epitopes sequences is available for many commercial proteins).

Protein Engineering to Reduce the Allergenicity, Cross-reactivity Effect of Proteins

The methods described thus far have led to identification of epitope areas on an acceptor protein, each containing epitope sequences. These subsets of amino acids are preferred for introducing mutations that are meant to modify the immunogenicity of the acceptor protein. An even more preferred subset of amino acids to target by mutagenesis are ‘hot spot amino acids’, which appear in several different epitope sequences, or which corresponds to anchor amino acids of the epitope patterns.

Thus, genetic engineering mutations should be designed in the epitope areas, preferably in epitope sequences, and more preferably in the ‘hot spot amino acids’.

Changing one or more of the amino acids defining the epitope area of the parent plant protein by genetic engineering mutations of a DNA sequence encoding the parent protein can be carried out using two different approaches: 1. gene replacement by gene targeting, where the target gene is Knock-out by homologous recombination (Kempin et al., Nature 389,802-803,1997) and replaced by the genetic engineered mutated gene also integrated by homologous recombination or 2. by site directed engineering of chromosomal plant genes by introducing specific chimeric oligonucleotides consisting of DNA and RNA stretches carrying the mutations (Zhu, T, Proc.Natl.Acad.Sci. USA, Vol. 96,8768-8773,1999)

Substitution, Deletion, Insertion

When the epitope area(s) have been identified, a protein variant exhibiting a modified immunogenicity may be produced by changing the identified epitope area of the parent protein by genetic engineering mutation of a DNA sequence encoding the parent protein.

The epitope identified may be changed by substituting at least one amino acid of the epitope area. In a preferred embodiment at least one anchor amino acid or hot spot amino acid is changed. The change will often be substituting to an amino acid of different size, hydrophilicity, and/or polarity, such as a small amino acid versus a large amino acid, a hydrophilic amino acid versus a hydrophobic amino acid, a polar amino acid versus a non-polar amino acid and a basic versus an acidic amino acid.

Other changes may be the addition or deletion of at least one amino acid of the epitope sequence, preferably deleting an anchor amino acid or a hot spot amino acid. Furthermore, an epitope pattern may be changed by substituting some amino acids, and deleting/adding other.

When one uses protein engineering to eliminate epitopes, it is indeed possible that new epitopes are created, or existing epitopes are duplicated. To reduce this risk, one can map the planned mutations at a given position on the 3-dimensional structure of the protein of interest, and control the emerging amino acid constellation against a database of known epitope patterns, to rule out those possible replacement amino acids, which are predicted to result in creation or duplication of epitopes. Thus, risk mutations can be identified and eliminated by this procedure, thereby reducing the risk of making mutations that lead to increased rather than decreased allergenicity.

Introduction of Consensus Sequences for Post-translational Modifications in the Epitope Areas

In another embodiment, the mutations are designed, such that recognition sites for post-translational modifications are introduced in the epitope areas, and the protein variant is expressed in a suitable host organism capable of the corresponding post-translational modification. These post-translational modifications may serve to shield the epitope and hence lower the immunogenicity of the protein variant relative to the protein backbone. Post-translational modifications include glycosylation, phosphorylation, N-terminal processing, acylation, ribosylation and sulfatation. A good example is N-glycosylation. N-glycosylation is found at sites of the sequence Asn-Xaa-Ser, Asn-Xaa-Thr, or Asn-Xaa-Cys, in which neither the Xaa residue nor the amino acid following the tri-peptide consensus sequence is a proline (T. E. Creighton, ‘Proteins—Structures and Molecular Properties, 2nd edition, W. H. Freeman and Co., New York, 1993, pp. 91-93). It is thus desirable to introduce such recognition sites in the sequence of the backbone protein. The specific nature of the glycosyl chain of the glycosylated protein variant may be linear or branched depending on the protein and the host cells. Another example is phosphorylation: The protein sequence can be modified so as to introduce serine phophorylation sites with the recognition sequence Arg-Arg-(Xaa)ₙ-Ser (where n=0, 1, or 2) (SEQ ID NOS: 3 and 4), which can be phosphorylated by the cAMP-dependent kinase or tyrosine phosphorylation sites with the recognition sequence -Lys/Arg-(Xaa)₃-Asp/Glu-(Xaa)₃-Tyr (SEQ ID NO: 5), which can usually be phophorylated by tyrosine-specific kinases (T. E. Creighton, “Proteins—Structures and molecular properties”, 2nd ed., Freeman, N.Y., 1993).

Randomized Approaches to Introduce Modifications in Epitope Areas

In order to generate protein variants, more than one amino acid residue may be substituted, added or deleted, these amino acids preferably being located in different epitope areas. In that case, it may be difficult to assess a priori how well the functionality of the protein is maintained while antigenicity is reduced, especially since the possible number of mutation-combinations becomes very large, even for a small number of mutations. In that case, it will be an advantage, to establish a library of diversified mutants each having one or more changed amino acids introduced and selecting those variants, which show good retention of function and at the same time a significant reduction in antigenicity.

A diversified library can be established by a range of techniques known to the person skilled in the art (Reetz M T; Jaeger K E, in ‘Biocatalysis—from Discovery to Application’ edited by Fessner W D, Vol. 200, pp. 31-57 (1999); Stemmer, Nature, vol. 370, p.389-391, 1994; Zhao and Arnold, Proc. Natl. Acad. Sci., USA, vol. 94, pp. 7997-8000, 1997; or Yano et al., Proc. Natl. Acad. Sci., USA, vol. 95, pp 5511-5515, 1998). These include, but are not limited to, ‘spiked mutagenesis’, in which certain positions of the protein sequence are randomized by carrying out PCR mutagenesis using one or more oligonucleotide primers which are synthesized using a mixture of nucleotides for certain positions (Lanio T, Jeltsch A, Biotechniques, Vol. 25(6), 958,962,964-965 (1998)). The mixtures of oligonucleotides used within each triplet can be designed such that the corresponding amino acid of the mutated gene product is randomized within some predetermined distribution function. Algorithms have been disclosed, which facilitate this design (Jensen L J et al., Nucleic Acids Research, Vol. 26(3), 697-702 (1998)).

In an embodiment substitutions are found by a method comprising the following steps: 1) a range of substitutions, additions, and/or deletions are listed encompassing several epitope areas (preferably in the corresponding epitope sequences, anchor amino aids, and/or hot spots), 2) a library is designed which introduces a randomized subset of these changes in the amino acid sequence into the target gene, e.g. by spiked mutagenesis, 3) the library is expressed, and preferred variants are selected. In another embodiment, this method is supplemented with additional rounds of screening and/or family shuffling of hits from the first round of screening (J. E. Ness, et al, Nature Biotechnology, vol. 17, pp. 893-896, 1999) and/or combination with other methods of reducing immunogenicity by genetic means (such as that disclosed in WO 92/10755).

The library may be designed, such that at least one amino acid of the epitope area is substituted. In a preferred embodiment at least one amino acid of the epitope sequence itself is changed, and in an even more preferred embodiment, one or more hot spot amino acids are changed. The library may be biased such that towards introducing an amino acid of different size, hydrophilicity, and/or polarity relative to the original one of the ‘protein backbone’. For example changing a small amino acid to a large amino acid, a hydrophilic amino acid to a hydrophobic amino acid, a polar amino acid to a non-polar amino acid or a basic to an acidic amino acid. Other changes may be the addition or deletion of at least one amino acid of the epitope area, preferably deleting an anchor amino acid. Furthermore, substituting some amino acids and deleting or adding others may change an epitope.

Diversity in the protein variant library can be generated at the DNA triplet level, such that individual codons are variegated e.g. by using primers of partially randomized sequence for a PCR reaction. Further, several techniques have been described, by which one can create a library with such diversity at several locations in the gene, which are too far apart to be covered by a single (spiked) oligonucleotide primer. These techniques include the use of in vivo recombination of the individually diversified gene segments as described in WO 97/07205 on page 3, line 8 to 29 or by using DNA shuffling techniques to create a library of full length genes that combine several gene segments each of which are diversified e.g. by spiked mutagenesis (Stemmer, Nature 370, pp. 389-391, 1994 and U.S. Pat. Nos. 5,605,793 and 5,830,721). In the latter case, one can use the gene encoding the “protein backbone” as a template double-stranded polynucleotide and combining this with one or more single or double-stranded oligonucleotides as described in claim 1 of U.S. Pat. No. 5,830,721. The single-stranded oligonucleotides could be partially randomized during synthesis. The double-stranded oligonucleotides could be PCR products incorporating diversity in a specific region. In both cases, one can dilute the diversity with corresponding segments containing the sequence of the backbone protein in order to limit the number of changes that are on average introduced. As mentioned above, methods have been established for designing the ratios of nucleotides (A; C; T; G) used at a particular codon during primer synthesis, so as to approximate a desired frequency distribution among a set of desired amino acids at that particular codon. This allows one to bias the partially randomized mutagenesis towards e.g. introduction of post-translational modification sites, chemical modification sites, or simply amino acids that are different from those that define the epitope or the epitope area. One could also approximate a sequence in a given location or epitope area to the corresponding location on a homologous, human protein.

Occasionally, one would be interested in testing a library that combines a number of known mutations in different locations in the primary sequence of the ‘protein backbone’. These could be introduced post-translational or chemical modification sites, or they could be mutations, which by themselves had proven beneficial for one reason or another (e.g. decreasing antigenicity, or improving specific activity, performance, stability, or other characteristics) . In such cases, it may be desirable to create a library of diverse combinations of known sequences. For example if 12 individual mutations are known, one could combine (at least) 12 segments of the ‘protein backbone’ gene in which each segment is present in two forms: one with and one without the desired mutation. By varying the relative amounts of those segments, one could design a library (of size 2¹²) for which the average number of mutations per gene can be predicted. This can be a useful way of combining elements that by themselves give some, but not sufficient effect, without resorting to very large libraries, as is often the case when using ‘spiked mutagenesis’. Another way to combine these ‘known mutations’ could be by using family shuffling of oligomeric DNA encoding the known changes with fragments of the full length wild type sequence.

d) Screening Protein Variants

Assays for Reduced Allergenicity

When protein variants have been constructed based on the methods described in this invention, it is desirable to confirm their antibody binding capacity, functionality, immunogenicity and/or allergenicity using a purified preparation. For that use, the protein variant of interest can be expressed in larger scale, purified by conventional techniques, and the antibody binding and functionality should be examined in detail using dose-response curves and e.g. direct or competitive ELISA (C-ELISA).

The potentially reduced allergenicity (which is likely, but not necessarily true for a variant w. low antibody binding) should be tested in in vivo or in vitro model systems: e.g. an in vitro assays for immunogenicity such as assays based on cytokine expression profiles or other proliferation or differentiation responses of epithelial and other cells incl. B-cells and T-cells. Further, animal models for testing allergenicity should be set up to test a limited number of protein variants that show desired characteristics in vitro. Useful animal models include the guinea pig intratracheal model (GPIT) (Ritz, et al. Fund. Appl. Toxicol., 21, pp. 31-37, 1993), mouse subcutaneous (mouse-SC) (WO 98/30682, Novo Nordisk), the rat intratracheal (rat-IT) (WO 96/17929, Novo Nordisk), and the mouse intranasal (MINT) (Robinson et al., Fund. Appl. Toxicol. 34, pp. 15-24, 1996) models.

The immunogenicity of the protein variant is measured in animal tests, wherein the animals are immunized with the protein variant and the immune response is measured. Specifically, it is of interest to determine the allergenicity of the protein variants by repeatedly exposing the animals to the protein variant by the intratracheal route and following the specific IgG and IgE titers. Alternatively, the mouse intranasal (MINT) test can be used to assess the allergenicity of protein variants. By the present invention the allergenicity is reduced at least 3 times as compared to the allergenicity of the parent protein, preferably 10 times reduced, more preferably 50 times.

However, the present inventors have demonstrated that the performance in ELISA correlates closely to the immunogenic responses measured in animal tests. To obtain a useful reduction of the allergenicity of a protein, the IgG, preferably IgE binding capacity of the protein variant must be reduced to at least below 75%, preferably below 50%, more preferably below 25% of the IgE binding capacity of the parent protein as measured by the performance in IgE ELISA, given the value for the IgE binding capacity of the parent protein is set to 100%.

Thus a first assessment of the immunogenicity and/or allergenicity of a protein can be made by measuring the antibody binding capacity or antigenicity of the protein variant using appropriate antibodies. This approach has also been used in the literature (WO 99/47680).

Determining Functionality

A wide variety of protein functionality assays are available in the literature. Especially, those suitable for automated analysis are useful for this invention.

1) Allergens with Enzyme Activity

Several have been published in the literature such as protease assays (WO 99/34011, Genencor International; J. E. Ness, et al, Nature Biotechn., 17, pp. 893-896, 1999), oxidoreductase assays (Cherry et al., Nature Biotechn., 17 , pp. 379-384, 1999, and assays for several other enzymes (WO 99/45143, Novo Nordisk). Those assays that employ soluble substrates can be employed for direct analysis of functionality of immobilized protein variants. Also enzyme inhibitors can be tested in this way.

2) Allergens with Ligand-binding Activities

Some of the allergens do not have enzyme activities, but are able to find specific molecules in a stoichiometric way. One such example is birch pollen allergen Bet v1, which has been shown to be a lipid binding protein. In general, allergens groups 12 and 13 include proteins with a strong homology to cytosolic fatty acid-binding proteins.

A number of allergens exhibit protein-binding capacities. Examples include allergens belonging to group 10 (Der f 10, Der p 10) and group 11 with a considerable homology to tropomyosin and paramyosin.

The impact of protein engineering on the functionality of the proteins belonging to this group can be assessed by simple ligand-binding studies (f.e. Scatchard plots) (In: Textbook of Biochemistry with clinical application, Thomas M Devlin, Ed, A Wiley Medical Publication, John Wiley & Sons, New York, Chichester, Brisbane, Toronto, Singapore).

3) Allergens Not Belonging to Any of These Groups

A number of allergens might not reveal an easily measurable activity. In these cases, the functionality of protein variants is assessed by evaluating the phenotypic appearance of the resulting plants.

e) Production of Transgenic Plants

Transgenic plants expressing the modified allergens have the purpose of substituting the original plant or animal for modified plants or animals. Methods for engineering of plants and animals are well known in the art. For example, for plants see Day, (1996) Crit. Rev. Food Sci. & Nut. 36(S), 549-567, which are incorporated herein by reference. See also Fuchs and Astwood (1996) Food Tech. 83-88. Methods for making recombinant animals are also well established. See, for example, Colman, A. “Production of therapeutic proteins in the milk of transgenic livestock” (1998) Biochem. Soc. Symp. 63, 141-147; Espanion ans Niemann, (1996) DTW Dtxch Tierarztl Wochenschr 103(8-9), 320-328; and Colman, Am. J. Clin. Nutr. 63(4), 639S-6455S, which are incorporated herein by reference.

The definition paragraphs above describe how to prepare the transgenic plants of the invention, i.e. plants transformed so as to produce the proteins as disclosed herein.

Materials and Methods

Materials

ELISA Reagents

Horse Radish Peroxidase labelled pig anti-rabbit-Ig (Dako, DK, P217, dilution 1:1000).

Rat anti-mouse IgE (Serotec MCA419; dilution 1:100).

Mouse anti-rat IgE (Serotec MCA193; dilution 1:200).

Biotin-labelled mouse anti-rat IgG1 monoclonal antibody (Zymed 03-9140; dilution 1:1000)

Biotin-labelled rat anti-mouse IgG1 monoclonal antibody (Serotec MCA336B; dilution 1:2000)

Streptavidin-horse radish peroxidase (Kirkegard & Perry 14-30-00; dilution 1:1000).

Buffers and Solutions

PBS (pH 7.2 (1 liter))

| | |
|---|---|
| NaCl | 8.00 g |
| KCl | 0.20 g |
| K₂HPO₄ | 1.04 g |
| KH₂PO₄ | 0.32 g |

Washing buffer PBS, 0.05% (v/v) Tween 20

Blocking buffer PBS, 2% (wt/v) Skim Milk powder

Dilution buffer PBS, 0.05% (v/v) Tween 20, 0.5% (wt/v) Skim Milk powder

Citrate buffer 0.1M, pH 5.0-5.2

Stop-solution (DMG-buffer)

Sodium Borate, borax (Sigma)

3,3-Dimethyl glutaric acid (Sigma)

Tween 20: Poly oxyethylene sorbitan mono laurate (Merck cat no. 822184)

PMSF (phenyl methyl sulfonyl flouride) from Sigma

Succinyl-Alanine-Alanine-Proline-Phenylalanine-paranitro-anilide (Suc-AAPF-pNP) Sigma no. S-7388, Mw 624.6 g/mol.

mPEG (Fluka)

Coloring Substrate

OPD: o-phenylene-diamine, (Kementec cat no. 4260)

Methods

Immunisation of Brown Norway Rats

Twenty intratracheal (IT) immunisations were performed weekly with 0.100 ml 0.9% (wt/vol) NaCl (control group), or 0.100 ml of a protein dilution (˜0.1-1 mg/ml). Each group contained 10 rats. Blood samples (2 ml) were collected from the eye one week after every second immunisation. Serum was obtained by blood clothing and centrifugation and analysed as indicated below.

Immunisation of Balb/C Mice

Twenty subcutaneous (SC) immunisations were performed weekly with 0.05 ml 0.9% (wt/vol) NaCl (control group), or 0.05 ml of a protein dilution (˜0.01-0.1 mg/ml). Each group contained 10 female Balb/C mice (about 20 grams) purchased from Bomholdtgaard, Ry, Denmark. Blood samples (0.100 ml) were collected from the eye one week after every second immunisation. Serum was obtained by blood clothing and centrifugation and analysed as indicated below.

ELISA Procedure for Detecting Serum Levels of IgE and IgG

Specific IgG1 and IgE levels were determined using the ELISA specific for mouse or rat IgG1 or IgE. Differences between data sets were analysed by using appropriate statistical methods.

Activation of CovaLink Plates:

A fresh stock solution of cyanuric chloride in acetone (10 mg/ml) is diluted into PBS, while stirring, to a final concentration of 1 mg/ml and immediately aliquoted into CovaLink NH2 plates (100 microliter per well) and incubated for 5 minutes at room temperature. After three washes with PBS, the plates are dried at 50° C. for 30 minutes, sealed with sealing tape, and stored in plastic bags at room temperature for up to 3 weeks.

Mouse anti-Rat IgE was diluted 200× in PBS (5 microgram/ml). 100 microliters were added to each well. The plates were coated overnight at 4° C.

Unspecific adsorption was blocked by incubating each well for 1 hour at room temperature with 200 microliters blocking buffer. The plates were washed 3× with 300 microliters washing buffer.

Unknown rat sera and a known rat IgE solution were diluted in dilution buffer: Typically 10×, 20× and 40× for the unknown sera, and ½ dilutions for the standard IgE starting from 1 μg/ml. 100 microliters were added to each well. Incubation was for 1 hour at room temperature.

Unbound material was removed by washing 3× with washing buffer. The anti-rat IgE (biotin) was diluted 2000× in dilution buffer. 100 microliters were added to each well. Incubation was for 1 hour at room temperature. Unbound material was removed by washing 3× with washing buffer.

Streptavidin was diluted 1000× in dilution buffer. 100 microliters were added to each well. Incubation was for 1 hour at room temperature. Unbound material was removed by washing 3× with 300 microliters washing buffer. OPD (0.6 mg/ml) and H₂O₂ (0.4 microliter/ml) were dissolved in citrate buffer. 100 microliters were added to each well. Incubation was for 30 minutes at room temperature. The reaction was stopped by addition of 100 microliters H₂SO₄. The plates were read at 492 nm with 620 nm as reference.

Similar determination of IgG can be performed using anti Rat-IgG and standard rat IgG reagents.

Similar determinations of IgG and IgE in mouse serum can be performed using the corresponding species-specific reagents.

Direct IgE Assay:

To determine the IgE binding capacity of protein variants one can use an assay, essentially as described above, but using sequential addition of the following reagents:

(1) Mouse anti-rat IgE antibodies coated in wells;

(2) Known amounts of rat antiserum containing igE against the parent protein;

(3) Dilution series of the protein variant in question (or parent protein as positive control);

(4) Rabbit anti-parent antibodies

(5) HRPO-labelled anti-rabbit Ig antibodies for detection using OPD as described.

The relative IgE binding capacity (end-point and/or affinity) of the protein variants relative to that of the parent protein are determined from the dilution-response curves. The IgE-positive serum can be of other animals (including humans that inadvertently have been senstitized to the parent protein) provided that the species-specific anti-IgE capture antibodies are changed accordingly.

Competitive ELISA (C-ELISA):

C-ELISA was performed according to established procedures. In short, a 96 well ELISA plate was coated with the parent protein. After proper blocking and washing, the coated antigen was incubated with rabbit anti-enzyme polyclonal antiserum in the presence of various amounts of modified protein (the competitior). The residual amount of rabbit antiserum was detected by horseradish peroxidase-labelled pig anti-rabbit immunoglobulin.

### EXAMPLES

### Example 1

### Identification of Epitope Sequences and Epitope Patterns

High diversity libraries (10¹²) of phages expressing random hexa-, nona- or dodecapetides as part of their membrane proteins, were screened for their capacity to bind purified specific rabbit IgG, and purified rat and mouse IgG1 and IgE antibodies. The phage libraries were obtained according to prior art (se WO 92/15679 hereby incorporated by reference).

The antibodies were raised in the respective animals by subcutaneous, intradermal, or intratracheal injection of relevant proteins dissolved in phosphate buffered saline (PBS). The respective antibodies were purified from the serum of immunised animals by affinity chromatography using paramagnetic immunobeads (Dynal AS) loaded with pig anti-rabbit IgG, mouse anti-rat IgG1 or IgE, or rat anti-mouse IgG1 or IgE antibodies.

The respective phage libraries were incubated with the IgG, IgG1 and IgE antibody coated beads. Phages, which express oligopeptides with affinity for rabbit IgG, or rat or mouse IgG1 or IgE antibodies, were collected by exposing these paramagnetic beads to a magnetic field. The collected phages were eluted from the immobilised antibodies by mild acid treatment, or by elution with intact enzyme. The isolated phages were amplified as know to the specialist. Alternatively, immobilised phages were directly incubated with E. coli for infection. In short, F-factor positive E. coli (e.g. XL-1 Blue, JM101, TG1) were infected with M13-derived vector in the presence of a helper-phage (e.g. M13K07), and incubated, typically in 2×YT containing glucose or IPTG, and appropriate antibiotics for selection. Finally, cells were removed by centrifugation. This cycle of events was repeated 2-5 times on the respective cell supernatants. After selection round 2, 3, 4, and 5, a fraction of the infected E. coli was incubated on selective 2×YT agar plates, and the specificity of the emerging phages was assessed immunologically. Thus, phages were transferred to a nitrocellulase (NC) membrane. For each plate, 2 NC-replicas were made. One replica was incubated with the selection antibodies, the other replica was incubated with the selection antibodies and the immunogen used to obtain the antibodies as competitor. Those plaques that were absent in the presence of immunogen, were considered specific, and were amplified according to the procedure described above.

The specific phage-clones were isolated from the cell supernatant by centrifugation in the presence of polyethyleneglycol. DNA was isolated, the DNA sequence coding for the oligopeptide was amplified by PCR, and the DNA sequence was determined, all according to standard procedures. The amino acid sequence of the corresponding oligopeptide was deduced from the DNA sequence.

Thus, a number of peptide sequences with specificity for the protein specific antibodies, described above, were obtained. These sequences were collected in a database, and analysed by sequence alignment to identify epitope patterns. For this sequence alignment, conservative substitutions (e.g. aspartate for glutamate, lysine for arginine, serine for threonine) were considered as one. This showed that most sequences were specific for the protein the antibodies were raised against. However, several cross-reacting sequences were obtained from phages that went through 2 selection rounds only. In the first round 22 epitope patterns were identified.

In further rounds of phage display, more antibody binding sequences were obtained leading to more epitope patterns. Further, the literature was searched for peptide sequences that have been found to bind environmental allergen-specific antibodies (J All Clin Immunol 93 (1994) pp. 34-43; Int Arch Appl Immunol 103 (1994) pp. 357-364; Clin Exp Allergy 24 (1994) pp. 250-256; Mol Immunol 29 (1992) pp. 1383-1389; J Immunol 121 (1989) pp. 275-280; J. Immunol 147 (1991) pp. 205-211; Mol Immunol 29 (1992) pp. 739-749; Mol Immunol 30 (1993) pp. 1511-1518; Mol Immunol 28 (1991) pp. 1225-1232; J. Immunol 151 (1993) pp. 7206-7213). These antibody binding peptide sequences were included in the database.

Table 1 below shows identified epitope patterns of Bet v1 (WO 99/47680). A mino acids are noted using the single letter code (G=glycine, A=alanine etc.) Multiple letters combined mean that in that specific position several amino acids awere recurrent. A capital means that the amino acid was more represented than the amino acid represented by a minor letter.

TABLE 1

| | aa1 | aa2 | aa3 | aa4 | aa5 | aa6 | aa7 | aa8 | aa9 | aa10 | aa11 | aa12 |
|---|---|---|---|---|---|---|---|---|---|---|---|---|
| 1 | ts | R | Y | — | kr | kr | P | — | L | | | |
| 2 | R | R | Y | P | St | — | Rk | al | st | | | |
| 3 | Y | I | — | K | L | | | | | | | |
| 4 | liag | — | Kr | Q | St | Y | Kr | | | | | |
| 5 | liv | ST | G | P | — | A | G | | | | | |
| 6 | P | — | S | D | A | G | | | | | | |
| 7 | P | Rk | St | D | P | G | | | | | | |
| 8 | D | P | R | D | T | G | | | | | | |
| 9 | F | H | V | D | K | P | Yt | A | al | Q | | |
| 10 | N | St | A | Rk | A | R | — | A | S | C | R | I |
| 11 | rk | R | F | — | N | N | — | E | L | | | |
| 12 | Rk | R | F | A | N | T | Ed | — | al | | | |
| 13 | de | Q | I | F | F | T | | | | | | |
| 14 | E | Y | | | | | | | | | | |
| 15 | fwy | P | als | P | A | P | — | S | | | | |
| 16 | Q | liv | Y | G | D | T | | | | | | |
| 17 | Ag | KR | I | D | P | Rk | | | | | | |
| 18 | dn | A | D | S | — | G | Yt | P | R | | | |
| 19 | S | R | S | A | | | | | | | | |
| 20 | L | St | G | R | S | S | | | | | | |
| 21 | lag | R | Q | — | — | Ed | | | | | | |
| 22 | P | S | Y | P | D | | | | | | | |
| 23 | P | S | I | — | C | | | | | | | |
| 24 | Ed | — | D | D | — | Rsky | | | | | | |
| 25 | AP | — | S | E | N | Rk | | | | | | |
| 26 | Ts | P | E | G | DE | | | | | | | |
| 27 | R | A | S | D | IL | ST | D | L | L | | | |
| 28 | E | DE | Rk | R | C | | | | | | | |
| 29 | P | W | S | W | | | | | | | | |
| 30 | rk | — | Q | R | Ed | E | D | | | | | |
| 31 | RDk | Qs | — | E | — | Q | agilv | | | | | |
| 32 | K | — | E | L | hknqr | gilv | | | | | | |
| 33 | L | Li | — | H | As | Li | G | | | | | |
| 34 | D | — | — | wf | R | N | — | L | | | | |
| 35 | V | de | A | A | F | | | | | | | |
| 36 | rk | S | V | Y | KQ | | | | | | | |
| 37 | st | sty | — | gvl | st | — | W | vi | | | | |
| 38 | I | M | S | — | L | ag | | | | | | |
| 39 | A | A | — | T | — | G | A | A | | | | |
| 40 | N | KR | L | A | Tsy | | | | | | | |

### Example 2

### Localization of Epitope Sequences and Epitope Areas on the 3D-structure of Acceptor Proteins

Epitope sequences were assessed on the 3D-structure of the protein of interest, using appropriate software (e.g. SwissProt Pdb Viewer, WebLite Viewer).

In a first step, the identified epitope patterns were fitted with the 3D-structure of the enzymes. A sequence of at least 3 amino acids, defining a specific epitope pattern, was localized on the 3D-structure of the acceptor protein. Conservative mutations (e.g. aspartate for glutamate, lysine for arginine, serine for threonine) were considered as one for those patterns for which phage display had evidenced such exchanges to occur. Among the possible sequences provided by the protein structure, only those were retained where the sequence matched a primary sequence, or where it matched a structural sequence of amino acids, where each amino acid was situated within a distance of 5 Å from the next one. Occasionally, the mobility of the amino acid side chains, as provided by the software programme, had to be taken in to consideration for this criterium to be fulfilled.

Secondly, the remaining anchor amino acids as well as the variable amino acids, i.e. amino acids that were not defining a pattern but were present in the individual sequences identified by phage library screening, were assessed in the area around the various amino acid sequences localized in step 1. Only amino acids situated within a distance of 5 Å from the next one were included.

Finally, an accessibility criterium was introduced. The criterium was that at least half of the anchor amino acids had a surface that was >20% accessible. Typically, 0-2 epitopes were retained for each epitope pattern. In some cases, two different amino acids could with equal probability be part of the epitope (e.g. two leucines located close to each other in the protein 3D-structure).

The percentage “surface accessible area” of an amino acid residue of the parent protein is defined as the Connolly surface (ACC value) measured using the DSSP program to the relevant protein part of the structure, divided by the residue total surface area and multiplied by 100. The DSSP program is disclosed in W. Kabsch and C. Sander, BIOPOLYMERS 22 (1983) pp. 2577-2637. The residue total surface areas of the 20 natural amino acids are tabulated in Thomas E. Creighton, PROTEINS; Structure and Molecular Principles, W. H. Freeman and Company, NY, ISBN: 0-7167-1566-X (1984).

Thus, a number of epitope sequences were identified and localized on the surface of various proteins. As suggested by sequence alignment of the antibody binding peptides, structural analysis confirmed most of the epitopes to be enzyme specific, with only few exceptions. Overall, most of the identified epitopes were at least partially structural. However, some proteins expressed predominantly primary sequence epitopes. Typically, the epitopes were localized in very discrete areas of the enzymes, and different epitope sequences often shared some amino acids (hot-spots).

The identified epitope sequences are shown below.

| | |
|---|---|
| Bet.v1-1.1 | T52 R70 Y81/Y83 K80 K103 L114 |
| Betv1-15.1 | F64 P63 L62 P59 A37 P35 S39/S40 |
| Betv1-40.1 | N159 R17 L18 A21 |

It is common knowledge that amino acids that surround binding sequences can affect binding of a ligand without participating actively in the binding process. Based on this knowledge, areas covered by amino acids with potential steric effects on the epitope-antibody interaction, were defined around the identified epitopes. Practically, all amino acids situated within 5 Å from the amino acids defining the epitope were included. The accessibility criterium was not included for defining epitope areas, as hidden amino acids can have an effect on the surrounding structures.

For Bet v1, the following amino acid residues belong to the epitope area that corresponds to each epitope sequence indicated above.

| | |
|---|---|
| Betv1-1.1 | T7 E8 T9 T10 L18 F19 F22 I23 I44 E45 G46 N47 G48 |
| | G49 P50 G51 T52 I53 K54 K68 D69 R70 V71 D72 E73 |
| | V74 D75 H76 N78 F79 K80 Y81 N82 Y83 S84 V85 I86 |
| | K97 I98 S99 N100 E101 I102 K103 I104 V105 S112 I113 |
| | L114 K115 I116 L144 |
| Betv1-15.1 | F30 P31 K32 V33 A34 P35 Q36 A37 I38 S39 S40 V41 E42 |
| | K55 I56 S57 F58 P59 E60 G61 L62 P63 F64 K65 Y66 G89 |
| | P90 M139 T142 L143 |
| Betv1-40.1 | S11 I13 P14 A15 A16 R17 L18 F19 A21 F22 I23 L24 D25 |
| | G26 F30 I104 S112 L114 L144 V147 E148 L151 D156 |
| | A157 Y158 N159 |

### Example 3

### Production, Selection, and Evaluation of Enzyme Variants with Reduced Antigenicity or Immunogenicity

Hot-spots or epitopes were mutated using techniques known to the expert in the field (e.g. site-directed mutagenesis, error-prone PCR).

Variants were made by the following procedures:

1) Site-directed mutagenesis of amino acids defining epitopes, with an effect on IgG1 and/or IgE responses in mice.

2) Site-directed mutagenesis of epitopes, with examples of epitope duplication, and new epitope formation, respectively, predicted by the epitope-database.

3) Site-directed mutagenesis of amino acids defining epitope areas, with a differential effect on IgG1 and IgE antibody levels in mice, and an inhibiting effect on IgG binding, respectively.

Amino acid exchanges giving new epitopes or duplicating existing epitopes according to the information collected in the epitope-database, were avoided in the mutagenesis process.

Enzyme variants were screened for reduced binding of antibodies raised against the backbone enzyme. This antibody binding was assessed by established assays (e.g. competitive ELISA, agglutination assay).

Variants with reduced antibody binding capacity were further evaluated in animal studies.

Mice were immunized subcutaneously weekly, for a period of 20 weeks, with 50 μl 0.9% (wt/vol) NaCl (control group), or 50 μl 0.9% (wt/vol) NaCl containing 10 μg of protein. Blood samples (100 μl) were collected from the eye one week after every second immunization. Serum was obtained by blood clotting and centrifugation.

Specific IgG1 and IgE levels were determined using the ELISA specific for mouse or rat IgG1 or IgE. Differences between data sets were analyzed by using appropriate statistical methods.

1. A method of preparing a transgenic plant expressing a protein variant having modified immunogenicity as compared to a parent protein, comprising the steps of:
(a) obtaining antibody binding peptide sequences involved in antibody binding,
(b) using the sequences to localize epitope sequences on the primary and/or the 3-dimensional structure of a parent protein,
(c) defining an epitope area including amino acids situated within 5 Å from the epitope amino acids constituting the epitope sequence,
(d) changing one or more of the amino acids defining the epitope area of the parent protein by genetic engineering mutations of a DNA sequence encoding the parent protein,
(e) introducing the mutated DNA sequence into a suitable host, culturing the host and expressing the protein variant,
(f) evaluating the immunogenicity of the protein variant using the parent protein as reference,
(g) introducing the mutated DNA sequence into an expression construct and transforming a suitable plant cell with the construct, and
(h) regenerating the plant from the plant cell.

2. The method of claim 1, wherein the sequences of step (a) are obtained by screening a random peptide display package library with antibodies raised against any protein of interest and sequencing the amino acid sequence of the antibody binding peptide, or the DNA sequence encoding the antibody binding peptide.

3. The method of claim 2, wherein antibodies for screening the random peptide display package library are raised against the protein allergen.

4. The method of claim 2, wherein the peptide display package library is a phage display library.

5. The method of claim 1, wherein the antibody binding peptide sequences of step (a) are obtained by screening a library of known peptides related to the primary sequence of any protein of interest, with antibodies raised against the protein of interest.

6. The method of claim 1, wherein epitope patterns are identified by sequence alignment of antibody binding peptide sequences and these epitope patterns are used to guide localization of epitope sequences on the 3-dimensional structure of the parent protein.

7. The method of claim 1, wherein the epitope area of step (c) equals the epitope sequence.

8. The method of claim 1, wherein hot spot amino acids of the parent protein are identified.

9. The method of claim 1, wherein the epitope area is changed by substituting, adding and/or deleting at least one amino acid.

10. The method of claim 1, wherein the epitope sequence is changed by substituting, adding and/or deleting at least one amino acid.

11. The method of claim 1, wherein the hot spot amino acids are changed by substituting, adding and/or deleting at least one amino acid.

12. The method of claims 9, wherein amino acids in the epitope area are changed by substituting and/or inserting at least one amino acid by an amino acid which render the substituted and/or inserted amino acid a target for in vivo posttranslational modification.

13. The method of claim 10, wherein amino acids in the epitope sequence are changed by substituting and/or inserting at least one amino acid by an amino acid which render the substituted and/or inserted amino acid a target for in vivo posttranslational modification.

14. The method of claim 11, wherein the hot spot amino acids are changed by substituting and/or inserting at least one amino acid by an amino acid which render the substituted and/or inserted amino acid a target for in vivo posttranslational modification.

15. The method of claim 9, wherein the amino acid for substitution and/or insertion is selected from the group consisting of K, C, D, E, Q, R and Y.

16. The method of claim 10, wherein the amino acid for substitution and/or insertion is selected from the group consisting of K, C, D, E, Q, R and Y.

17. The method of claim 11, wherein the amino acid for substitution and/or insertion is selected from the group consisting of K, C, D, E, Q, R and Y.

18. The method of claim 1, wherein the immunogenicity is measured by antibody binding assays.

19. The method of claim 1, wherein the protein variant has reduced allergenicity.

20. The method of claim 19, wherein the allergenicity of the protein variant is less than 75% of the allergenicity of the parent protein.

21. The method of claim 20, wherein the allergenicity of the protein variant is less than 50% of the allergenicity of the parent protein.

22. The method of claim 21, wherein the allergenicity of the protein variant is less than 25% of the allergenicity of the parent protein.

23. The method of claim 1, wherein the parent protein is an environmental allergen.

24. The method of claim 23, wherein the parent protein is a food allergen.

25. The method of claim 1, wherein the host cell in step e) is a bacteria, fungal or plant cell.

26. The method of claim 25, wherein if the host in step e) is a bacteria or a fungal cell, the evaluating of the immunogenicity in step f) should be carried out on protein expressed by a plant cell.

27. A transgenic plant and a seed thereof transformed with a nucleotide sequence encoding a protein allergen having modified immunogenicity as compared to a parent protein.

28. The plant of claim 27, wherein the protein allergen is selected from the group consisting of food allergens.

29. The plant of claim 27, wherein the protein allergen is modified by changing the epitope area, epitope sequence or hot spot amino acids by substituting, adding and/or deleting at least one amino acid.

30. The plant of claim 29, wherein amino acids in the epitope area, the epitope sequence or the hot spot amino acids are changed by substituting and/or inserting at least one amino acid by an amino acid which render the substituted and/or inserted amino acid a target for in vivo posttranslational modification.

31. The plant of claim 29, wherein the amino acid for substitution and/or insertion is selected from the group consisting of K, C, D, E, Q, R and Y.

32. A DNA construct comprising a DNA sequence encoding a protein variant having modified immunogenicity as compared to a parent protein.

33. An expression vector comprising a DNA construct of claim 32.

34. A host cell transformed with the expression vector of claim 33.
